(19) United States
(12) Patent Application Publication
(10) Pub. No.: US 2008/0050319 A1
(43) Pub. Date: Feb. 28, 2008

(54) Benzylidene-Alpha-Dicarbonyl Compounds as Novel Uv Absorbers

(75) Inventors: Oskar Koch, Gottingen (DE); William Johncock, Reinbek (DE)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: SYMRISE GMBH & CO. KG, Holzminden (DE)

(21) Appl. No.: 11/568,606

(22) PCT Filed: Apr. 26, 2005
(86) PCT No.: PCT/EP05/51865

§ 371 Date: Sep. 18, 2007

(30) Foreign Application Priority Data
May 3, 2004 (DE) 10 2004 021 805.6
Jun. 17, 2004 (DE) 10 2004 029 239.6

(51) Int. Cl. A61K 8/37; A61K 8/35; A61Q 17/04

(52) U.S. Cl. 424/59

(57) ABSTRACT

The use of a compound of the formula 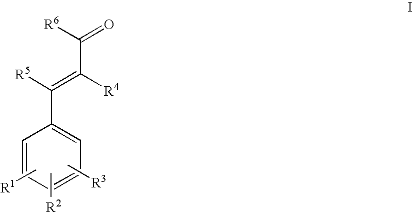 wherein
R¹-R³ independently of one another are hydrogen, C₁-C₈-alkyl or C₁-C₈-alkoxy,
R⁴ is COR, CO₂R, CONR₂, where R is C₁-C₈-alkyl or C₃-C₈-cycloalkyl
R⁵ is H or C₁-C₈-alkyl,
R⁶ is aryl, aryl substituted by up to three C₁-C₈-alkyl- or C₁-C₈-alkoxy, or C₃-C₈-cycloalkyl

as UV filters in cosmetic formulations, in particular in combination with UV filters from the group consisting of methoxycinnamate derivatives and/or dibenzoylmethane derivatives, with the proviso that R⁵ is H if R⁴ is COR, is described.

The invention relates to the use of certain benzylidene-β-dicarbonyl compounds as UV filters (UV absorbers) in cosmetic and dermatological formulations, such as sunscreen compositions and day and hair care products. The invention furthermore relates to corresponding cosmetic and dermatological formulations. Finally, the invention also relates to certain novel benzylidene-β-dicarbonyl compounds which can be employed as UV-B filters.

UV rays are classified according to their wavelength into UV-A rays (320-400 nm) and UV-B rays (280-320 nm). The harmful action, the occurrence of sunburn (erythema), is caused here decisively by the UV-B radiation. Dermatological studies have shown that UV-A radiation also causes skin damage. This range of the radiation is thus held responsible for premature ageing of skin up to skin cancer.

From this finding, for a modern cosmetic sunscreen it is essential to cover the two ranges, both UV-A and UV-B. For this reason, in addition to the UV-B absorbers which are already known, such as e.g. camphor derivatives, salicylic acid derivatives, benzophenones, cinnamates, benzimidazoles and triazines, novel UV-A absorbers have been developed. One of the most important representatives is 4-dimethylethyl-4′-methoxydibenzoylmethane.

A disadvantage is, however, that incompatibilities sometimes occur when combinations of UV-A with UV-B absorbers are employed in sunscreen compositions. Thus e.g. a combination of 4-dimethylethyl-4′-methoxydibenzoylmethane and octyl methoxycinnamate is not photostable because of interactions. The consequence of this is that the protective performance under sunlight decreases rapidly.

The primary object of the present invention was therefore to provide UV filters (UV absorbers), and indeed in particular UV-B filters, which not only are photostable by themselves, but moreover with other UV filters form cosmetic formulations which are compatible in respect of photostability. In this context, the solubility of the UV filters in conventional cosmetic oils should preferably be high.

This primary object was achieved according to the invention by the use of the compounds of the formula I

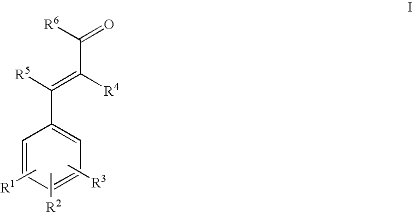

wherein
R¹-R³ independently of one another are hydrogen, C₁-C₈-alkyl or C₁-C₈-alkoxy,
R⁴ is COR, CO₂R, CONR₂, where R is C₁-C₈-alkyl (preferably) or C₃-C₈-cycloalkyl
R⁵ is H or C₁-C₈-alkyl,
R⁶ is aryl, aryl substituted by up to three C₁-C₈-alkyl- or C₁-C₈-alkoxy, or C₃-C₈-cycloalkyl

as UV filters (preferably UV-B filters) in cosmetic or dermatological formulations.

In this context, there is the proviso that

R⁵ is H if R⁴ is COR.

In the context of this text, compounds are shown with the aid of their pictorial formulae; regardless of the pictorial representation chosen in the individual case, each pictorial formula here includes all the possible configuration isomers of the compound shown and mixtures thereof; in particular, the possible E/Z isomers (and mixtures thereof) are also included, and in the case of the presence of chiral centres, the R and the S enantiomers (and mixtures thereof) are in each case also included.

A process for the preparation of a cosmetic or dermatological formulation, wherein an active amount of one or more compounds of the formula I (as defined above) is mixed with further constituents of a cosmetic or dermatological formulation corresponds to this use according to the invention.

A method for protecting skin or hair against UV radiation, in particular UV-B radiation, wherein an active amount of one or more compounds of the formula I (as defined above) is applied to the skin or hair in the form of a cosmetic or dermatological formulation which comprises further constituents furthermore corresponds to the use according to the invention.

The compounds of the formula I to be used according to the invention are suitable in particular for combination with UV filters from the group consisting of methoxycinnamate derivatives and/or dibenzoylmethane derivatives; such combinations have proved to be surprisingly photostable.

Compounds according to the invention are indeed described in DE 10 87 902 A1 in the form of a (very broad) general formula, but only for industrial UV protection, so that there are significant differences in respect of the particular technical field and the particular requirements. The incorporation (and fixing) of an industrial UV filter into plastics, films, fibres or the like is thus somewhat completely different to the incorporation into a cosmetic formulation (e.g. an O/W emulsion), which is applied to the skin and can lead to penetration through the skin—depending on the chemical structure of the UV filter.

DE 19755650 A1 discloses substituted alpha-methyl-styrene derivatives which can be employed as photostable UV filters. In this context, the compounds disclosed always include an alpha-methyl group and two identical terminal groups (designated R³ and R⁴). In contrast to the compounds to be employed according to the invention, the compounds disclosed are UV-A filters.

It was thus surprising that the compounds of the formula I according to the invention are outstanding UV-B filters and in a very particular manner result in photostable mixtures (with other UV filters).

Compounds of the above formula I which are particularly preferred for use as UV filters in cosmetic or dermatological formulations are those wherein
R¹-R³ independently of one another are hydrogen, C₁-C₈-alkyl or C₁-C₈-alkoxy,
R⁴ is CO₂R, where R is C₁-C₈-alkyl,
R⁵ is H or C₁-C₈-alkyl,
R⁶ is aryl or aryl substituted by up to three C₁-C₈-alkyl- or C₁-C₈-alkoxy.

In contrast to the compounds where R⁴ is COR or CONR₂, the compounds mentioned which are preferably to be employed are quite readily soluble in cosmetic oils. While the solubility in cosmetic oils, such as e.g. isopropyl myristate, Miglyol-812 or Witconol TN, of the compounds which are preferably to be employed is regularly greater than 10 wt. %, based on the total weight of the resulting mixture (solution), the solubility for the compounds where R⁴ is COR or CONR₂ is regularly lower than 10 wt. %.

The use of compounds of the formula I wherein
R¹ is methoxy,
R² and R³ are H,
R⁴ is CO₂R, where R is C₂-C₅-alkyl and
R⁶ is phenyl or phenyl substituted by up to three C₁-C₈-alkyl- or C₁-C₈-alkoxy

is particularly preferred.

The use of these compounds leads to particularly photostable mixtures, in this context compare also the examples below.

For reasons of a particularly good solubility again, the use of compounds in which
R¹ is methoxy which is located in the para position to the radical carrying the substituents R₄, R₅ and R₆,
R² and R³ are hydrogen,
R⁴ is CO₂R, where R is C₄-C₅-alkyl (preferably n-butyl),
R⁵ is H or C₁-C₈-alkyl, and
R⁶ is phenyl

is particularly preferred.

In contrast to the otherwise identical compounds where R is C₁-C₃-alkyl, those where R is C₄-C₅-alkyl are liquids (and not solids), so that when the particularly preferred compounds are employed, there is no risk of crystallizing out in the finished cosmetic or dermatological formulation.

The following compounds are preferred for use as UV filters for the UV-B range:

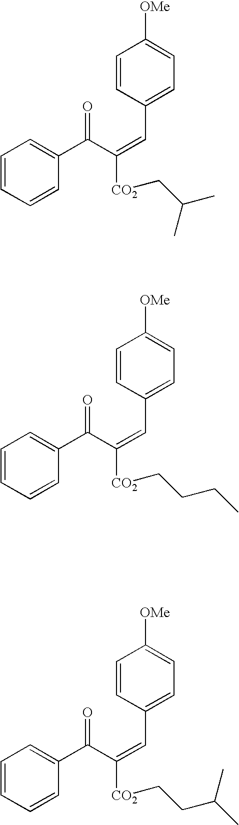

Compounds of the above formula I wherein R⁶ is C₃-C₈-cycloalkyl, preferably cyclohexyl, are particularly preferred for use as UV filters in cosmetic or dermatological formulations. Particularly photostable formulations result when they are used.

In this case, preferably, in particular to achieve a good solubility, R⁴ is CO₂R, where R is C₁-C₈-alkyl, preferably R is methyl or ethyl. If R is ethyl, the solubility is particularly good.

The benzylidene-β-dicarbonyl compounds to be used according to the invention can be used as UV filters (UV absorbers) in cosmetic or dermatological formulations, in particular for protection against acute (sunburn) and chronic (premature ageing of the skin) skin damage especially in sunscreen compositions, day care products and hair care products.

The benzylidene-β-dicarbonyl compounds to be used according to the invention can be employed individually or in a mixture in the corresponding formulations (preparations); they can also be employed in combination with UV absorbers of other substance classes or also with these in any desired mixtures with one another.

A cosmetic or dermatological formulation (preparation) according to the invention comprises
one or more compounds of the formula I as defined above (having the general or a preferred meaning of the substituents), as UV absorbers.

A preferred cosmetic or dermatological formulation furthermore comprises
one or more further UV absorbers, in particular from the group consisting of methoxycinnamate derivatives (p-methoxycinnamic acid esters) and/or dibenzoylmethane derivatives

and/or
coated or non-coated pigments of metal oxides.

These aspects are explained further in detail in the following, particularly preferred embodiments also being described. In the cosmetic or dermatological formulations. the UV absorbers and pigments employed are preferably chosen and co-ordinated in their particular amount relative to one another such that they co-operate such that the sun protection factor of the formulation is increased synergistically.

The UV absorbers employed are particularly advantageously chosen and co-ordinated in their particular amount relative to one another such that the critical wavelength of the formulation λ_{crit.} is >380 nm. In this context, the critical wavelength is the wavelength at the integral of the spectral absorption curve reaches 90% of the integral of 290-400 nm.

The following UV absorbers with which the compounds of the formula I can be combined may be mentioned by way of example:
p-Aminobenzoic acid
p-Aminobenzoic acid ethyl ester (25 mol) ethoxylated
p-Dimethylaminobenzoic acid 2-ethylhexyl ester
p-Aminobenzoic acid ethyl ester (2 mol) N-propoxylated
p-Aminobenzoic acid glycerol ester
Salicylic acid homomenthyl ester (homosalate) (Neo-Heliopan®HMS)
Salicylic acid 2-ethylhexyl ester (Neo-Heliopan®OS)
Triethanolamine salicylate
4-Isopropylbenzyl salicylate
Anthranilic acid menthyl ester (Neo Heliopan®MA)
Diisopropylcinnamic acid ethyl ester
p-Methoxycinnamic acid 2-ethylhexyl ester (Neo Heliopan® AV)
Diisopropylcinnamic acid methyl ester
p-Methoxycinnamic acid isoamyl ester (Neo Heliopan®E 1000)
p-Methoxycinnamic acid diethanolamine salt
p-Methoxycinnamic acid isopropyl ester
2-Ethylhexyl 2-cyano-3,3-diphenylacrylate (Neo Heliopan®303)
Ethyl 2-cyano-3,3′-diphenylacrylate
2-Phenylbenzimidazolesulfonic acid and salts (Neo Heliopan®Hydro)
3-(4′-Trimethylammonium)-benzylidene-bornan-2-one methyl sulfate
Terephthalylidene-dibornanesulfonic acid and salts (Mexoryl®SX)
4-t-Butyl-4′-methoxy-dibenzoylmethane (avobenzone)/(Neo Heliopan®357)
β-Imidazole-4(5)-acrylic acid (urocanic acid)
2-Hydroxy-4-methoxybenzophenone (Neo Heliopan®BB)
2-Hydroxy-4-methoxybenzophenone-5-sulfonic acid
Dihydroxy-4-methoxybenzophenone
2,4-Dihydroxybenzophenone
Tetrahydroxybenzophenone
2,2′-Dihydroxy-4,4′-dimethoxybenzophenone
2-Hydroxy-4-n-octoxybenzophenone
2-Hydroxy-4-methoxy-4′-methylbenzophenone
3-(4′-Sulfo)benzylidene-bornan-2-one and salts
3-(4′-Methylbenzylidene)-d,l-camphor (Neo Heliopan®MBC)
3-Benzylidene-d,l-camphor
4-Isopropyldibenzoylmethane
2,4,6-Trianilino-(p-carbo-2′-ethylhexyl-1′-oxy)-1,3,5-triazine
Phenylene-bis-benzimidazyl-tetrasulfonic acid disodium salt (Neo Heliopan®AP)
2,2′-(1,4-Phenylene)-bis-(1H-benzimidazole-4,6-disulfonic acid), monosodium salt
N-[(2 and 4)-[2-(oxoborn-3-ylidene)methyl]benzyl]-acrylamide polymer
Phenol,-(2H-benzotriazol-2-yl)-4-methyl-6-(2-methyl-3(1,3,3,3-tetramethyl-1-(trimethylsilyl)-oxy)-disiloxyanyl)-propyl), (Mexoryl®XL)
4,4′-[(6-[4-(1,1 -Dimethyl)-aminocarbonyl)-phenylamino]-1,3,5-triazine-2,4-diyl)diimino]-bis-(benzoic acid 2-ethylhexyl ester) (Uvasorb®HEB)
2,2′-Methylene-bis-(6-(2H-benzotriazol-2-yl)-4-1,1,3,3-tetramethylbutyl)-phenol), (Tinosorb®M)
2,4-bis-[4-(2-ethylhexyloxy)-2-hydroxyphenyl]-1,3,5-triazine
Benzylidenemalonate-polysiloxane (Parsol®SLX)
Glyceryl ethylhexanoate dimethoxycinnamate
Disodium 2,2′-dihydroxy-4,4′-dimethoxy-5,5′-disulfo-benzophenone
Dipropylene glycol salicylate
Sodium hydroxymethoxybenzophenone sulfonate
4,4′,4-(1,3,5-Triazine-2,4,6-triyltriimino)-tris-benzoic acid tris(2-ethylhexyl ester) (Uvinul®T150)
2,4-Bis-[{(4-(2-Ethyl-hexyloxy)-2-hydroxy}-phenyl]-6-(4-methoxyphenyl)-1,3,5-triazine, (Tinosorb®S)
2,4-Bis-[{(4-(3-sulfonato)-2-hydroxy-propyloxy)-2-hydroxy}-phenyl]-6-(4-methoxyphenyl)-1,3,5-triazine sodium salt
2,4-Bis-[{(3-(2-Propyloxy)-2-hydroxy-propyloxy)-2-hydroxy}-phenyl]-6-(4-methoxy-phenyl)-1,3,5-triazine
2,4-Bis-[{4-(2-Ethyl-hexyloxy)-2-hydroxy}-phenyl]-6-[4-(2-methoxyethylcarbonyl)-phenylamino]-1,3,5-triazine
2,4-Bis-[{4-(3-(2-propyloxy)-2-hydroxy-propyloxy)-2-hydroxyl-phenyl]-6-[4-(2-ethylcarbonxyl)-phenylamino]-1,3,5-triazine
2,4-Bis-[{4-(2-Ethyl-hexyloxy)-2-hydroxy}-phenyl]-6-(1-methyl-pyrrol-2-yl-)-1,3,5-triazine
2,4-Bis-[{4-tris-(trimethylsiloxy-silylpropyloxy)-2-hydroxy}-phenyl]-6-(4-methoxyphenyl)-1,3,5-triazine
2,4-Bis-[{4-(2″-Methylpropenyloxy)-2-hydroxy}-phenyl]-6-(4-methoxyphenyl)-1,3,5-triazine
2,4-Bis-[{4-(1′,1′,1′,3′5′,5′,5′-Heptamethylsiloxy-2″-methyl-propyloxy)-2-hydroxy}-phenyl]-6-(4-methoxyphenyl)-1,3,5-triazine
2-(4-Diethylamino-2-hydroxybenzoyl)-benzoic acid hexyl ester (Uvinul® A Plus)
Indanylidene compounds according to DE 100 55 940 (=WO 02/38537)

In this context, UV absorbers which are particularly suitable for combination are
p-Aminobenzoic acid
3-(4′-Trimethylammonium)-benzylidene-bornan-2-one methyl sulfate
Salicylic acid homomenthyl ester (Neo-Heliopan®HMS)
2-Hydroxy-4-methoxy-benzophenone (Neo Heliopan®BB)
2-Phenylbenzimidazolesulfonic acid (Neo Heliopan®Hydro)
Terephthalylidene-dibornanesulfonic acid and salts (Mexoryl®SX)
4-tert-Butyl-4′-methoxydibenzoylmethane (Neo Heliopan®357)
3-(4′-Sulfo)benzylidene-bornan-2-one and salts
2-Ethylhexyl 2-cyano-3,3-diphenylacrylate (Neo Heliopan®303)
N-[(2 and 4)-[2-(oxoborn-3-ylidene)methyl]benzyl]-acrylamide polymer
p-Methoxycinnamic acid 2-ethylhexyl ester (Neo Heliopan®AV)
p-Aminobenzoic acid ethyl ester (25 mol) ethoxylated
p-Methoxycinnamic acid isoamyl ester (Neo Heliopan®E 1000)
2,4,6-Trianilino-(p-carbo-2′-ethylhexyl-1′-oxy)-1,3,5-triazine (Uvinal®T150)
Phenol,2-(2H-benzotriazol-2-yl)-4-methyl-6-(2-methyl-3(1,3,3,3-tetramethyl-1-(trimethylsilyl)-oxy)-disiloxyanyl)-propyl), (Mexoryl®XL)
4,4′-[(6-[4-(1,1-Dimethyl)-aminocarbonyl)-phenylamino]-1,3,5-triazine-2,4-diyl)-diimino]-bis-(benzoic acid 2-ethylhexyl ester) (UvasorbHEB)
3-(4′-Methylbenzylidene)-d,l-camphor (Neo Helipan®MBC)
3-Benzylidenecamphor
Salicylic acid 2-ethylhexyl ester (Neo Helipan®OS)
4-Dimethylaminobenzoic acid 2-ethylhexyl ester (padimate O)
Hydroxy-4-methoxy-benzophenone-5-sulfonic acid and Na salt
2,2′-Methylene-bis-(6-(2H-benztriazol-2-yl )-4-1,1,3,3-tetramethylbutyl)-phenol), (Tinosorb®M)
Phenylene-bis-benzimidazyl-tetrasulfonic acid disodium salt (Neo Heliopan®AP)
2,4-Bis-[{(4-(2-Ethyl-hexyloxy)-2-hydroxy}-phenyl]-6-(4-methoxyphenyl)-1,3,5-triazine, (Tinosorb®S)
Benzyl idenemalonate-polysiloxane (Parsol®SLX)
Menthyl anthranilate (Neo Heliopan®MA)
2-(4-Diethylamino-2-hydroxybenzoyl)-benzoic acid hexyl ester (Uvinul® A Plus)
Indanylidene compounds according to DE 100 55 940 (=WO 02/38537)

It may also be advantageous to use polymer-bonded or polymeric UV absorbers in formulations according to the invention, in particular those such as are described in WO-A-92/20690. The combination of the benzylidene-β-dicarbonyl compounds to be used according to the invention with finely divided inorganic and organic pigments, such as e.g. titanium dioxide, zinc oxide and iron oxide or Tinosorb®M, in sunscreen and day care products with UV protection is likewise possible.

The list of UV filters mentioned which can be employed in combination with the benzylidene-β-dicarbonyl compounds of the formula I in the context of the present invention is of course not conclusive.

The total amount of all the (mono- or poly-)sulfonated water-soluble UV filter substances in the finished cosmetic or dermatological formulations, for example of phenylene-bis-benzimidazyl-tetrasulfonic acid disodium salt and salts thereof and/or the corresponding disulfonic acid and salts thereof and/or 2-phenylbenzimidazole-5-sulfonic acid and salts thereof and/or 2-hydroxy-4-methoxybenzophenone-5-sulfonic acid and salts thereof and/or 4-(2-oxo-3-bornylidenemethyl)-benzenesulfonic acid and salts thereof and/or 2-methyl-5-(2-oxo-3-bornylidene-methyl)-benzenesulfonic acid and salts thereof and/or benzene-1,4-di-(2-oxo-3-bornylidenemethyl)-10-sulfonic acid and salts thereof, is advantageously chosen from the range of from 0.1 to 10.0 wt. %, preferably 0.5 to 6.0 wt. %, based on the total weight of the formulations, if the presence of these substances is desired.

The total amount of oil-soluble UV filter substances (including the compounds of the formula I) in the finished cosmetic or dermatological formulations, for example of 4,4′,4″-(1,3,5-triazine-2,4,6-triyltriimino)-tris-benzoic acid tris-(2-ethylhexyl ester) and/or 4-tert-butyl-4′-methoxy-dibenzoylmethane and/or 4-methylbenzylidenecamphor and/or octyldimethyl-p-aminobenzoic acid and/or Mexoryl®XL and/or Uvasorb®HEB and/or Tinosorb®S and/or benzophenone-3 and/or Parsol®SLX and/or Neo Heliopan®MA is advantageously chosen from the range of from 0.1 to 10.0 wt. %, preferably 0.5 to 6.0 wt. %, based on the total weight of the formulations, if the presence of these substances is desired.

The total amount of 2-ethylhexyl p-methoxy-cinnamates and/or p-methoxycinnamic acid isoamyl ester in the finished cosmetic or dermatological formulations is advantageously chosen from the range of from 0.1 to 15.0 wt. %, preferably 0.5 to 7.5 wt. %, based on the total weight of the formulations, if the presence of these substances is desired.

The total amount of ethylhexyl 2-cyano-3,3-diphenylacrylate in the finished cosmetic or dermatological formulations, if the presence of this substance is desired, is advantageously chosen from the range of from 0.1 to 15.0 wt. %, preferably 0.5 to 10.0 wt. %, based on the total weight of the formulations.

The total amount of one or more salicylic acid derivatives in the finished cosmetic or dermatological formulations is in many cases advantageously chosen from the range of from 0.1 to 15.0 wt. %, preferably 0.5 to 10.0 wt. %, based on the total weight of the formulations. If ethylhexyl salicylate is chosen, it is advantageous to choose the total amount thereof from the range of from 0.1 to 5.0 wt. %. If homomenthyl salicylate is chosen, it is advantageous to choose the total amount thereof from the range of from 0.1 to 10.0 wt. %.

The particularly preferred combinations of (a) p-methoxycinnamic acid esters (methoxycinnamates) and/or dibenzoylmethane derivatives and (b) compounds of the formula I can be formulated in a photostable manner by employing from e.g. 0.1 to 5 wt. %, preferably 1 to 3 wt. % of 4-tert-butyl-4′-methoxydibenzoylmethane, 0.1 to 10 wt. %, preferably 1 to 7.5 wt. % of p-methoxycinnamic acid ethylhexyl or isoamyl ester and at least 0.2 wt. %, preferably 1 to 6 wt. % of the compounds of the formula I to be employed according to the invention.

Preferably, a ratio in the range of 1 part of dibenzoylmethane derivative, 2,5-3.5 parts of p-methoxycinnamic acid ester and 1.5-2.5 parts of the benzylidene-β-dicarbonyl compounds to be employed according to the invention is established. The combination of 1 part of dibenzoylmethane derivative, 3 parts of p-methoxycinnamic acid ester and 2 parts of the benzylidene-β-dicarbonyl compounds to be employed according to the invention is particularly preferred.

It is moreover advantageous to add to this three-component combination one or further very photostable UV absorbers, such as e.g. methylbenzylidenecamphor, 2-ethylhexyl 2-cyano-3,3′-diphenylacrylate, octyltriazone, Uvasorb®HEB, Tinosorb®S, Tinosorb®M, ethylhexyl salicylate, homomenthyl salicylate and phenylenebenzimidazolesulfonic acid or phenylene-bis-benzimidazole-tetrasulfonic acid disodium salt, Mexoryl®SX, Mexoryl®XL, Parsol®SLX, Uvinul®A Plus or indanylidene compounds according to DE 100 55 940.

Surprisingly, in cosmetic or dermatological formulations a synergistic increase in the sun protection factor is achieved by the use of benzylidene-β-dicarbonyl compounds of the formula I in combination with other UV filters. Example of a synergistic increase in the sun protection factor are cosmetic or dermatological emulsions which comprises both a compound of the formula I and etylhexyl methoxycinnamate or octocrylene, or a combination of a compound of the formula I with ethylhexyl methoxycinnamate and 2-phenylbenzimidazolesulfonic acid, or ethylhexyl methoxycinnamate and methylbenzylidenecamphor, or ethylhexyl methoxycinnamate and 4-t-butyl-4′-methoxy-dibenzoylmethane, or Neo Heliopan®AP and ethylhexyl methoxycinnamate, or a combination of compound of the formula I with octocrylene, methylbenzylidenecamphor and zinc oxide. Combinations of compound of the formula I with dibenzoylmethanes, methylbenzylidenecamphor, 2-phenylbenzimidazolesulfonic acid, Neo Heliopan®AP, Mexoryl®SX, Mexoryl®XL, Parsol®SLX, Tinosorb®S, Tinosorb®M, Uvinul®T150, Uvasorb®HEB, Uvinul®A Plus or indanylidene compounds according to DE 100 55 940 and microfine pigments, zinc oxide and titanium dioxide likewise show synergistic increases in the sun protection factor. The UV filter combinations mentioned are given here only by way of example; a synergistic effect also occurs in combination with other UV filters. Thus, all the particularly suitable UV absorbers (UV filters) already mentioned above and all UV filters approved in the following publications can be employed, individually or in combination with one another, in combination with compounds of the formula I.

| | |
|---|---|
| USA: | Food and Drug Administration (FDA). Publication |
| | in the Monograph for Sunscreen Drug Products for |
| | Over-The-Counter Human Use. |
| Europe: | Council Directive 76/768 EEC for approximation |
| | of the legal provisions of the member states |
| | relating to cosmetic agents to technical progress. |
| | Publications in the Official Journal of European |
| | Communities. |
| Japan: | Publication of the cosmetics directive of the |
| | Ministry of Health and Welfare (MHW). |
| Germany: | Publication in the legislation on cosmetic agents |
| | in accordance with the Foodstuffs and Commodities |
| | Act (LMBG). |
| Australia: | Registration by Therapeutic Goods Administration |
| | (TGA) and publication in the Australian Register |
| | of Therapeutic Goods (ARTG). |

A synergistic increase in the UV sun protection factor is regularly achieved by the combinations mentioned.

The combination of compounds of the formula I with UV-A absorbers results in an optimum broad-band protection performance (290-400 nm). A combination of compounds of the formula I with Neo Heliopan® AP (UV-AII absorber) and indanylidene compounds according to DE 100 55 940 (UV-AI absorber) may be mentioned in particular for this broad UV protection performance. Further UV-A filters which are preferred in combination with compounds of the formula I, by themselves or in a combination of compounds of the formula I and Neo Heliopan®AP and/or indanylidene compounds according to DE 100 55 940 are Mexoryl®SX, Mexoryl®XL, Tinosorb®M Tinosorb®S, benzophenone-3, benzophenone-4, Neo Heliopan®357, Neo Heliopan®MA and Uvinul®A Plus.

By combination of compounds of the formula I with Neo Heliopan®AP and a UV-B filter, e.g. etylhexyl methoxycinnamate or UV-B filter mixtures and coated or non-coated finely disperse metal oxides, such as e.g. zinc oxide, titanium dioxide, a UV broad-band protection performance is achieved with a critical wavelength λ_{crit.} of >380 nm (in this context cf. Diffey in Int. J. Cosm. Science 16, 47 (1994)).

Some of the benzylidene-β-dicarbonyl compounds of the formula I to be used according to the invention are (crystalline) solids under standard conditions (25° C.; 1,013 mbar) and must therefore be adequately dissolved in cosmetic formulations in order to avoid the problem of recrystallization after a relatively long storage time (in this context cf. the above comments on compounds of the formula I which are particularly preferred since they are liquid). Advantageously, to avoid recrystallization, an adequate amount of the oil components, liquid oil-soluble UV absorbers or alcohols conventionally employed in cosmetic formulations is employed, e.g. ethanol, isopropanol or 1-butanol. The use of the following oil components and/or UV absorbers is particularly preferable to achieve an adequate solubility of crystalline benzylidene-β-dicarbonyl compounds of the formula I to be used according to the invention.

Ethylhexyl methoxycinnamate, isoamyl methoxycinnamate, octocrylene, ethylhexyl salicylate, homosalate, menthyl anthranilate, padimate O, diisopropyl adipate, C₁₂₋₁₅-alkyl benzoate (Witconol TN), butylene glycol dicaprylate/dicaprate (Miglyol 8810), cocoglycerides (Myritol 331), capryl/capr. triglycerides (Miglyol 812), cetearyl iso-nonanate (Cetiol SN), PVP/hexadecene copolymer (Unimer U151), adipic acid/diethylene glycol/isononanoic acid copolymer (Lexorez 100), propylene glycol dicaprylate/dicaprate (Myritol PC), hexyl laurate (Cetiol A), dicapryl ether (Cetiol OE), diethylhexyl naphthalate (Hallbrite®TQ), butyloctyl salicylate (Hallbrite®BHB), dibutyl adipate (Cetiol B), triethyl citrate (Hydagen CAT), propylene glycol dibenzoate (Finsolv PG 22), tributyl citrate, dioctyl malate (Ceraphyl 45), dipropylene glycol dibenzoate (Benzoflex 245), acetyltributyl citrate (Citroflex A-4), acetyltriethyl citrate (Citroflex A-2). The list of oils mentioned which can be employed in the context of the present invention is of course not conclusive.

The total amount employed of all the components of the oily phase in cosmetic emulsions comprising compounds of the formula I is preferably in the range of from 0.5 to 30%, preferably 2 to 15%. All the abovementioned oil components and liquid oil-soluble UV filters are excellent solvents for all the crystalline oil-soluble UV absorbers.

It is a great disadvantage if UV absorbers leave behind stains which can no longer be washed out on items of clothing. In particular, it is known of the UV-A absorber tert-butylmethoxydibenzoylmethane that it causes stains on textiles which can no longer be washed out. The benzylidene-β-dicarbonyl compounds to be used according to the invention do not have this disadvantage, since any stains on textiles can be very readily washed out.

Sunscreen products should be water-resistant, so that UV adequate protection is ensured for the user, in particular children, during swimming or bathing. The compounds to be used according to the invention meet these requirements to a particular degree. In an O/W emulsion comprising 3% of a compound of the benzylidene-β-dicarbonyl compounds according to the invention, 97% substantivity of the UV absorber was measured after washing, and in a W/O emulsion the figure was 95%. The water resistance of sunscreen products comprising water-soluble, mono- or polysulfonated UV filters, such as e.g. Neo Heliopan®AP, Mexoryl®SX, benzophenone-4, Neo Heliopan®Hydro, and/or the oil-soluble UV absorbers listed above can furthermore be increased significantly by combination with compounds of the formula I.

It may furthermore be of considerable advantage to combine the UV absorbers mentioned according to the invention with chelating substances, such as are listed e.g. in EP-A 496 434, EP-A 313 305 and WO-94/04128, or with polyaspartic acid and ethylenediamine-tetramethyl-phosphonic acid salts.

The invention furthermore provides the use of the compounds of the formula I, which are to be used according to the invention, in combination with conventional UV absorbers to intensify the protection against harmful UV radiation beyond the extent of the protection which is achieved when employing the same total amounts, by themselves, of conventional UV filters or UV filters to be used according to the invention (synergistic effect).

The total amount of UV filter substances (UV-A, UV-B and/or broad-band filters) in the cosmetic or dermatological formulations according to the invention, whether as the individual substance or in any desired mixtures with one another, is advantageously in the range of from 0.1 to 30 wt. %, preferably 0.1 to 10.0 wt. %, in particular 0.5 to 5.0 wt. %, based on the total weight of the formulations.

Cosmetic and dermatological formulations according to the invention furthermore advantageously, although not necessarily, comprise inorganic pigments based on finely disperse metal oxides and/or other metal compounds which are sparingly soluble or insoluble in water, in particular the oxides of titanium (1102), zinc (ZnO), iron (e.g. Fe₂O₃), zirconium (ZrO₂). Silicon (SiO₂). Manganese (e.g. MnO), aluminium (A1₂O₃), cerium (e.g. Ce₂O₃), mixed oxides of the corresponding metals and blends of such oxides. These pigments are amorphous to X-rays or not amorphous to X-rays. They are particularly preferably pigments based on TiO₂.

Oxide pigments which are amorphous to X-rays are metal oxides or semi-metal oxides which reveal no or no detectable crystal structure in X-ray diffraction experiments. Such pigments are often obtainable by flame reactions, for example by reacting a metal halide or semi-metal halide with hydrogen and air (or pure oxygen) in a flame.

Oxide pigments are which are amorphous to X-rays are employed in cosmetic, dermatological or pharmaceutical formulations as thickening and thixotropy agents, flow auxiliaries for stabilizing emulsions and dispersions and as a carrier substance (for example for increasing the volume of finely divided powders or dusts). Known oxide pigments which are amorphous to X-rays and are often used in the formulation of cosmetic or dermatological compositions are, for example, highly pure silicon oxide. Highly pure silicon dioxide pigments which are amorphous to X-rays and have a particle size in the range of from 5 to 40 nm and an active surface area (BET) in the range of from 50 to 400 m²/g, preferably 150 to 300 m²/g, are preferred, the particles being regarded as spherical particles having very uniform dimensions. Macroscopically, the silicon dioxide pigment are detectable as loose, white powders. Silicon dioxide pigment are marketed commercially under the name Aerosil® (CAS No. 7631-85-9) or Carb-O-Sil Advantageous Aerosil® types are, for example, Aerosil®0X50. Aerosil®130, Aerosil®150, Aerosil®200, Aerosil®300, Aerosil®380, Aerosil®MQX 80. Aerosil®MOX 170, Aerosil®COK 84, Aerosil®R 202, Aerosil®R 805, Aerosil®R 812, Aerosil®R 972, Aerosil®R 974, Aerosil®R976.

Cosmetic or dermatological sunscreen formulations according to the invention advantageously comprise 0.1 to 20 wt. %, advantageously 0.5 to 10 wt. %, very particularly preferably 1 to 5 wt. % of oxide pigments which are amorphous to X-rays.

According to the invention, the inorganic pigments which are not amorphous to X-rays are advantageously in a hydrophobic form, i.e. they have been given a water-repellent treatment on the surface. This surface treatment can comprise providing the pigments with a thin hydrophobic layer by processes which are known per se. Such a process comprises, for example, generating the hydrophobic surface layer by a reaction in accordance with
nTiO2+m(RO)3Si—R′→nTiO2(surf.)

In this equation, n and m are stoichiometric parameters which are to be employed as desired, R and R′ are the desired organic radicals. Hydrophobized pigments prepared, for example, analogously to DE-A 33 14 742 are of advantage.

TiO₂ pigments such as are marketed by Degussa under the trade name T805 may be mentioned as examples. TiO₂/Fe₂O₃ mixed oxides such as are likewise available, for example, from Degussa under the trade name T817 are also preferred.

The total amount of inorganic pigments, in particular hydrophobic inorganic micropigments, in the finished cosmetic or dermatological formulations is advantageously in the range of from 0.1 to 30 wt. %, preferably 0.1 to 10.0, in particular 0.5 to 6.0 wt. %, based on the total weight of the formulations.

The cosmetic and/or dermatological formulations according to the invention can have a conventional composition and can serve for cosmetic and/or dermatological sun protection, and furthermore for the treatment, care and cleansing of the skin and/or hair and as a make-up product in decorative cosmetics. The formulations according to the invention can correspondingly be used, for example, depending on their build-up, as skin protection cream, cleansing milk, sunscreen lotion, nutrient cream, day or night cream etc. It is possible and advantageous, where appropriate, to use the formulations according to the invention as a base for pharmaceutical formulations. Those cosmetic and dermatological formulations which are in the form of a skin care or make-up product are preferred in particular. Creams, gels, lotions, alcoholic and aqueous/alcoholic solutions, emulsions or stick preparations are a typical embodiment. These compositions can furthermore comprise as further auxiliary substances and additives mild surfactants, co-emulsifiers, superfatting agents, pearlescent waxes, agents for imparting consistency, thickeners, polymers, silicone compounds, fats, waxes, stabilizers, biogenic active compounds, deodorizing active compounds, antidandruff agents, film-forming agents, swelling agents, hydropic agents, preservatives, insect repellents, tanning agents, artificial self-tanning agents (e.g. dihydroxyacetone), solubilizers, perfume oils, dyestuffs, germ-inhibiting agents and the like.

For use, the cosmetic and dermatological formulations according to the invention are applied to the skin and/or hair in a sufficient amount in the conventional manner for cosmetics. Those cosmetic and/or dermatological formulations according to the invention which are in the form of a cosmetic composition for protection of the skin and hair are particularly preferred. These can advantageously comprise at least one inorganic pigment, preferably an inorganic micropigment, in addition to UV-A, UV-B and/or broad-band filters used according to the invention.

The cosmetic and/or dermatological formulations according to the invention can comprise cosmetic auxiliary substances such as are conventionally used in such formulations, e.g. preservatives, bactericides, perfumes, substances for preventing foaming, dyestuffs, pigments which have a colouring action, thickeners, moisturizing and/or moisture-retaining substances, fats, oils, waxes or other conventional constituents of a cosmetic or dermatological formulation, such as alcohols, polyols, polymers, foam stabilizers, electrolytes, organic solvents or silicone derivatives. Possible nonionic emulsifiers or dispersing agents are the group formed by polyglyceryl 2-dipolyhydroxystearate (Dehymuls®PGPH), polyglyceryl 3-diiso-stearate (Lameform®TGI), polyglyceryl 4-isostearate (Isolan®GI 34), polyglyceryl 3-oleate, diisostearyl polyglyceryl 3-diisostearate (Isolan®PDI), polyglyceryl 3-methylglucose distearate (Tego Carey®450), polyglyceryl 3-beeswax (Cera Bellina®), polyglyceryl 4-caprate (polyglycerol caprate T2010/90), polyglyceryl 3-cetyl ether (Chimexane®NL), polyglyceryl 3-distearate (Cremophor®GS 32), polyglyceryl 2-stearate (Hostacerin®DGMS) and polyglyceryl polyricinoleate (Admul®WOL 1403) and mixtures thereof.

The particular amounts of cosmetic or dermatological auxiliary and carrier substances and perfume to be employed can be easily determined according to the nature of the particular product by simple trials by the person skilled in the art.

An additional content of antioxidants is in general preferred. According to the invention, all the antioxidants which are suitable or usual for cosmetic and/or dermatological uses can be used as favourable antioxidants.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. a-carotene, b-carotene, lycopene) and derivatives thereof, chlorogenic acid and derivatives thereof, liponic acid and derivatives thereof (e.g. dihydroliponic acid), aurothioglucose, propyl-thiouracil and other ihiols (e.g. thioredoxin, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) as well as sulfoximine compounds (e.g. buthionine sulfoximine, homocysteine sulfoximine, buthionine sulfones, penta-, hexa-, heptathionine sulfoximine) in very low tolerated dosages (e.g. pmol to μmol/kg), furthermore (metal) chelators (e.g. α-hydroxy-fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate) as well as coniferylbenzoate of benzoin resin, rutic acid and derivatives thereof, α-glycosylrutin, ferulic acid, furfurylideneglucitol, carnosine, butylhydroxytoluene, butylhydroxyanisole, nordihydroguaiac resin acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenium methionine), stilbenes and derivatives thereof (e.g. stilbene oxide, trans-stilbene oxide) and the derivatives suitable according to the invention (salts, esters, ethers, sugars, nucleotides, nucleosides, peptides and lipids) of these active compounds mentioned.

The amount of the abovementioned antioxidants (one or more compounds) in the formulations is preferably 0.001 to 30 wt. %, particularly preferably 0.05 to 20 wt. %, in particular 1 to 10 wt. %, based on the total weight of the formulation.

If vitamin E and/or derivatives thereof are the antioxidant or antioxidants, it is advantageous to choose the particular concentrations thereof from the range of from 0.001 to 10 wt. %, based on the total weight of the formulation.

If vitamin A or vitamin A derivatives, or carotenes or derivatives thereof are the antioxidant or antioxidants, it is advantageous to choose the particular concentrations thereof from the range of from 0.001 to 10 wt. %, based on the total weight of the formulation.

The lipid phase can advantageously be chosen from the following substance group:
mineral oils, mineral waxes;
oils, such as triglycerides of capric or caprylic acid, and furthermore natural oils, such as e.g. castor oil;
fats, waxes and other natural and synthetic fat substances, preferably esters of fatty acids with alcohols of low C number, e.g. with isopropanol, propylene glycol or glycerol, or esters of fatty alcohols with alkanoic acids of low C number or with fatty acids;
alkyl benzoates;
silicone oils, such as dimethylpolysiloxane, diethylpolysiloxane, diphenylpolysiloxane and mixed forms thereof.

The oily phases of the emulsions, oleogels or hydrodispersions or lipodispersions in the context of the present invention advantageously comprise substances from the group consisting of esters of saturated and/or unsaturated, branched and/or unbranched alkanecarboxylic acids having a chain length of from 3 to 30 C atoms and saturated and/or unsaturated, branched and/or unbranched alcohols having a chain length of from 3 to 30 C atoms, from the group consisting of esters of aromatic carboxylic acids and saturated and/or unsaturated, branched and/or unbranched alcohols having a chain length of from 3 to 30 C atoms. Such ester oils can then advantageously be chosen from the group consisting of isopropyl myristate, palmitate, stearate, oleate, n-butyl stearate, n-hexyl laurate, n-decyl oleate, isooctyl stearate, iso-nonyl stearate, isononyl isononanate, 2-ethylhexyl palmitate, ethylhexyl laurate, 2-hexyl-decyl stearate, 2-octyldodecyl palmitate, oleyl oleate, oleyl erucate, erucyl oleate and synthetic, semi-synthetic and natural mixtures of such esters, e.g. jojoba oil.

The oily phase can furthermore advantageously be chosen from the group consisting of branched and unbranched hydrocarbons and hydrocarbon waxes, silicone oils, dialkyl ethers, the group consisting of saturated or unsaturated, branched or unbranched alcohols, and the fatty acid triglycerides, namely the triglycerol esters of saturated and/or unsaturated, branched and/or unbranched alkanecarboxylic acids having a chain length of from 8 to 24, in particular 12 to 18 C atoms. The fatty acid triglycerides can advantageously be chosen, for example, from the group consisting of synthetic, semi-synthetic and natural oils, e.g. olive oil, sunflower oil, soya oil, groundnut oil, rapeseed oil, almond oil, palm oil, coconut oil, palm kernel oil and more of the like.

Any desired blends of such oil and wax components can also advantageously be employed in the context of the present invention. It may also be advantageous, where appropriate, to employ waxes, for example cetyl palmitate, as the sole lipid component (which is not UV-absorbing to a relevant extent) of the oily phase.

The oily phase advantageously comprises one or more substances from the group chosen from the group consisting of 2-ethylhexyl isostearate, ocryldodecanol, isotridecyl isononanoate, isoeicosane, 2-ethylhexyl cocoate, C₁₂₋₁₅-alkyl benzoate, caprylic/capric acid triglyceride, dicapryl ether.

Mixtures of C₁₂₋₁₅-alkyl benzoate and 2-ethylhexyl isostearate, mixtures of C₁₂₋₁₅-alkyl benzoate and isotridecyl isononanoate and mixtures of C₁₂₋₁₅-alkyl benzoate, 2-ethylhexyl isostearate and isotridecyl isononanoate are particularly advantageous.

The oily phase can also advantageously have a content of cyclic or linear silicone oils, it nevertheless being preferable to use an additional content of other oily phase components in addition to the silicone oil or silicone oils.

Cyclomethicone (octamethylcyclotetrasiloxane) can advantageously be employed as a silicone oil to be used. However, other silicone oils are also advantageously to be used in the context of the present invention, for example hexamethylcyclotrisiloxane, polydimethylsiloxane, poly(methylphenylsiloxane).

Mixtures of cyclomethicone and isotridecyl isononanoate and of cyclomethicone and 2-ethylhexyl isostearate are furthermore particularly advantageous.

The aqueous phase of the formulations according to the invention optionally advantageously comprises alcohols, diols or polyols (lower alkyl) and ethers thereof, preferably ethanol, isopropanol, propylene glycol, glycerol, ethylene glycol monoethyl or monobutyl ether, propylene glycol monomethyl, monoethyl or monobutyl ether, diethylene glycol monomethyl or monoethyl ether and analogous products, and furthermore alcohols (lower alkyl), e.g. ethanol, 1,2-propanediol and glycerol, and, in particular, one or more thickeners, which can advantageously be chosen from the group consisting of silicon dioxide, aluminium silicates, polysaccharides and derivatives thereof, e.g. hyaluronic acid, xanthan gum and hydroxypropylmethylcellulose, particularly advantageously from the group consisting of polyacrylates, preferably a polyacrylate from the group consisting of the so-called Carbopols, for example Carbopols of the types 980, 981, 1382, 2984 and 5984, in each case individually or in combination. A comprehensive description of the raw materials and active compounds employed in cosmetic compositions is given in DE 199 19 630 A1.

The invention also relates to novel compounds of the formula I

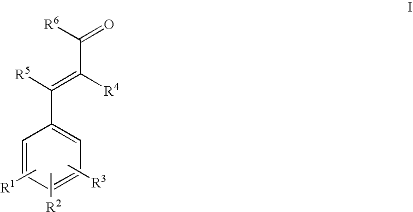

and in particular those wherein
R¹ is C₁-C₈-alkoxy which is located in the para position to the radical carrying the substituents R⁴, R⁵ and R⁶, preferably methoxy,
R² and R³ are hydrogen,
R⁴ is CO₂R, where R is C₁-C₈-alkyl,
R⁵ is H,
R⁶ is phenyl or cyclohexyl.

In this context, preferred compounds are (a) those in which
R⁴ is CO₂R, where R is methyl, ethyl, n-propyl, iso-propyl, n-butyl, sec-butyl, iso-butyl, tert-butyl, iso-amyl, preferably n-butyl, and
R⁶ is phenyl.

However, preferred compound are also (b) those in which
R⁴ is CO₂R, where R is methyl, ethyl, n-propyl, iso-propyl, n-butyl, sec-butyl, iso-butyl or tert-butyl, preferably ethyl, and
R⁶ is cyclohexyl.

Further preferred embodiments of the invention emerge from the following examples (including recipe examples, examples for the photostability etc.) and the attached patent claims.

### EXAMPLES FOR THE PREPARATION AND THE ABSORPTION PROPERTIES

### Example 1

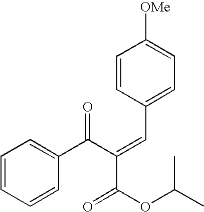

1 mol benzoylacetic acid isopropyl ester and 1 mol anisaldehyde are brought together in 200 g toluene, and 0.1 mol ammonium acetate and 0.1 mol propionic acid are added. The mixture is heated to the boiling point and the water formed is sluiced out via a water separator. After 4 h hours, the mixture is cooled to RT and washed with 100 g water and the product is distilled as an E/Z mixture (86:14). Yield: 60% of th. Extinction: 640 at 316 nm

### Example 2

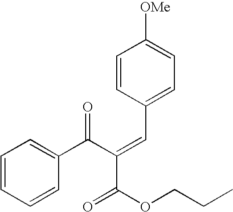

1 mol benzoylacetic acid n-propyl ester are reacted analogously to Example 1. Yield: 60% of th. (E/Z mixture 86:14) Extinction: 660 at 316 nm

### Example 3

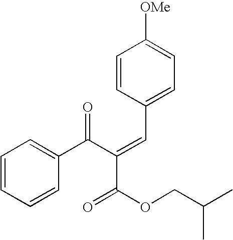

1 mol benzoylacetic acid isobutyl ester are reacted analogously to Example 1. Yield: 60% of th. (E/Z mixture 80:20) Extinction: 640 at 316 nm

### Example 4

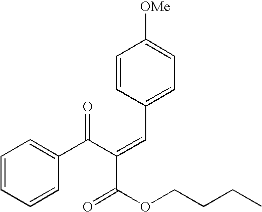

1 mol benzoylacetic acid n-butyl ester are reacted analogously to Example 1. Yield: 60% of th. (E/Z mixture 85:15) Extinction: 660 at 316 nm

### Example 5

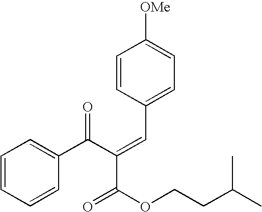

1 mol benzoylacetic acid isoamyl ester are reacted analogously to Example 1. Yield: 60% of th. (E/Z mixture 86:14) Extinction: 630 at 316 nm

### Example 6

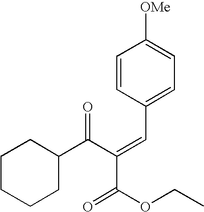

1 mol 3-cyclohexyl-3-oxo-propionic acid ethyl ester are reacted analogously to Example 1. Yield: 60% of th. (E/Z mixture 70:30) Extinction: 700 at 316 nm

### Example 7

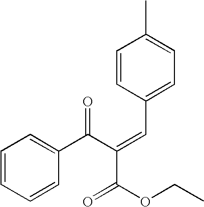

1 mol benzoylacetic acid ethyl ester are reacted with p-tolylaldehyde analogously to Example 1. Yield: 60% of th. (E/Z mixture 84:16) Extinction: 690 at 292 nm

### Example 8

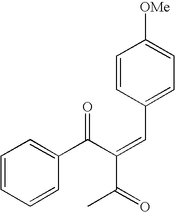

1 mol benzoylacetone are reacted analogously to Example 1. Yield: 60% of th. (E/Z mixture 15:85). The pure Z isomer is obtained as a solid (m.p. 76° C.) by recrystallization from methanol, extinction: 816 at 323 nm

### Example 9

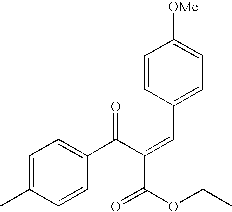

1 mol p-methylbenzoyacetic acid ethyl ester are reacted with anisaldehyde analogously to Example 1. Yield: 60% of th. (E/Z mixture 86:14) Extinction: 660 at 316 nm

### Recipe Example 1

Sunscreen Soft Cream (O/W), In Vitro SPF 3, Water-Resistant

| Part | Raw materials | INCI name | % (wt.) |
|---|---|---|---|
| A | Crodafos MCA | Cetyl Phosphate | 1.50 |
| | Cutina MD | Glyceryl Stearate | 2.00 |
| | Copherol 1250 | Tocopherylacetate | 0.50 |
| | Lanette 16 | Cetyl Alcohol | 1.00 |
| | Tegosoft TN | C12-15 Alkyl Benzoate | 24.00 |
| | Prisorine 3505 | Isostearic Acid | 1.00 |
| | UV filter compound | | 3.00 |
| | according to | | |
| | formula I | | |
| B | Water, dist. | Water (Aqua) | 59.60 |
| | EDETA B liq. | Tetrasodium EDTA | 0.20 |
| | Glycerol, 99% | Glycerin | 3.00 |
| | Phenoxyethanol | Phenoxyethanol | 0.70 |
| | Solbrol M | Methylparaben | 0.20 |
| | Solbrol P | Propylparaben | 0.10 |
| | Carbopol ETD | Carbomer | 0.20 |
| | 2050 | | |
| C | Sodium hydroxide | Sodium Hydroxide | 2.70 |
| | solution, 10% aq. | | |
| D | Perfume oil | Perfume (Fragrance) | 0.30 |

Preparation process
Part A: Heat to approx. 85° C.
Part B: Weigh out the raw materials, excluding Carbopol. Disperse in Carbopol with an Ultra Turrax. Heat to approx. 85° C. Add B to A.
Part C: Add immediately to A/B and then homogenize while hot (Ultra Turrax). Allow to cool, while stirring.
Part D: Add and stir.

### Recipe Example 2

Sunscreen Lotion (O/W), In Vitro SPF 20

| Part | Raw materials | INCI name | % (wt.) |
|---|---|---|---|
| A | Crodafos MCA | Cetyl Phosphate | 1.50 |
| | Cutina MD | Glyceryl Stearate | 2.00 |
| | Copherol 1250 | Tocopherylacetate | 0.50 |
| | Lanette 16 | Cetyl Alcohol | 1.00 |
| | Tegosoft TN | C12-15 Alkyl Benzoate | 10.60 |
| | Prisorine 3505 | Isostearic Acid | 1.00 |
| | UV filter compound | | 2.00 |
| | according to | | |
| | formula I | | |
| | Neo Heliopan ® AV | Ethyl Hexyl | 5.00 |
| | | Methoxycinnamate | |
| B | Water, dist. | Water (Aqua) | 55.07 |
| | EDETA B liq. | Tetrasodium EDTA | 0.20 |
| | Glycerol, 99% | Glycerin | 3.00 |
| | Phenoxyethanol | Phenoxyethanol | 0.70 |
| | Solbrol M | Methylparaben | 0.20 |
| | Solbrol P | Propylparaben | 0.10 |
| | Carbopol ETD 2050 | Carbomer | 0.20 |
| C | Sodium hydroxide | Sodium Hydroxide | 3.30 |
| | solution, | | |
| | 10% aq. | | |
| | Neo Heliopan ® | Phenylbenzimidazole | 13.33 |
| | Hydro, 15% | Sulfonic Acid | |
| | strength solu- | | |
| | tion neutralized | | |
| | with NaOH | | |
| D | Perfume oil | Perfume (Fragrance) | 0.30 |

Preparation process
Part A: Heat to approx. 85° C.
Part B: Weigh out the raw materials, excluding Carbopol. Disperse in Carbopol with an Ultra Turrax. Heat to approx. 85° C. Add B to A.
Part C: Add immediately to A/B and then homogenize while hot (Ultra Turrax). Allow to cool, while stirring.
Part D: Add and stir.

### Recipe Example 3

Sunscreen Milk (O/W), In Vitro SPF 6

| Part | Raw materials | INCI name | % (wt.) |
|---|---|---|---|
| A | Tegin M | Glyceryl Stearate | 2.50 |
| | Tagat S | PEG-30 Glyceryl | 1.95 |
| | | Stearate | |
| | Lanette O | Cetearyl Alcohol | 2.20 |
| | Copherol 1250 | Tocopherylacetat | 0.50 |
| | Miglyol 8810 | Butylene Glycol | 12.00 |
| | | Dicaprylate/Caprate | |
| | Tegsoft TN | C12-C15 Alkyl Benzoate | 8.00 |
| | Phenonip | Phenoxyethanol (and) | 0.15 |
| | | Methylparaben (and) | |
| | | Butylparaben (and) | |
| | | Ethyl-paraben | |
| | | (and) Propylparaben | |
| | UV filter | | 5.00 |
| | compound according | | |
| | to formula I | | |
| B | Water, dist. | Water (Aqua) | 43.90 |
| | EDETA BD | Disodium EDETA | 0.10 |
| | 1,2-Propylene | Propylene Glycol | 2.00 |
| | glycol | | |
| | | Phenoxyethanol (and) | 0.30 |
| | | Methylparaben (and) | |
| | | Butylparaben (and) | |
| | | Ethylparaben | |
| | | (and) Propylparaben | |
| C | Water, dist. | Water (Aqua) | 19.00 |
| | Carbopol 2050 | Carbomer | 0.40 |
| | NaOH, 10% strength | Sodium Hydroxide | 1.70 |
| D | Perfume oil | Perfume (Fragrance) | 0.30 |

Preparation process
Part A: Heat to 80-85° C.
Part B: Heat to 80-85° C., add part B to part A, while stirring.
Part C: Disperse the Carbopol in the water and neutralize with NaOH, while stirring. Add part C at approx. 60° C., while stirring. Allow to cool to RT (room temperature, 25° C.).
Part D: Add and stir.

### Recipe Example 4

Sunscreen Lotion (O/W), In Vitro SPF 21

| Part | Raw materials | INCI name | % (wt.) |
|---|---|---|---|
| A | Tegin M | Glyceryl Stearate | 2.50 |
| | Tagat S | PEG-30 Glyceryl | 1.95 |
| | | Stearate | |
| | Lanette O | Cetearyl Alcohol | 2.20 |
| | Copherol 1250 | Tocopherylacetat | 0.50 |
| | Miglyol 8810 | Butylene Glycol | 12.00 |
| | | Dicaprylate/Caprate | |
| | Tegosoft TN | C12-C15 Alkyl Benzoate | 8.00 |
| | Phenonip | Phenoxyethanol (and) | 0.20 |
| | | Methylparaben (and) | |
| | | Butylparaben | |
| | | (and) Ethylparaben (and) | |
| | | Propylparaben | |
| | UV filter | | 2.00 |
| | compound according | | |
| | to formula I | | |
| | Neo Heliopan ® AV | Ethylhexyl | 5.00 |
| | | Methoxycinnamate | |
| | Neo Heliopan ® 357 | Butyl Methoxydiben- | 1.00 |
| | | zoylmethane | |
| B | Water, dist. | Water (Aqua) | 39.35 |
| | EDETA BD | Disodium EDETA | 0.10 |
| | 1,2-Propylene | Propylene Glycol | 2.00 |
| | glycol | | |
| | Phenonip | Phenoxyethanol (and) | 0.30 |
| | | Methylparaben (and) | |
| | | Butylparaben | |
| | | (and) Ethylparaben (and) | |
| | | Propylparaben | |
| | Vitamin C | Ascorbic Acid | 0.10 |
| C | Water, dist. | Water (Aqua) | 20.00 |
| | Carbopol 2050 | Carbomer | 0.40 |
| | NaOH, 10% strength | Sodium Hydroxide | 1.70 |
| D | Perfume oil | Perfume (Fragrance) | 0.30 |

Preparation process
Part A: Heat to 80-85° C.
Part B: Heat to 80-85° C., add part B to part A, while stirring.
Part C: Disperse the Carbopol in the water and neutralize with NaOH, while stirring.
Part C add at approx. 60° C., while stirring. Allow to cool to RT.
Part D: Add and stir.

### Recipe Example 5

Sunscreen Lotion (O/W), In Vitro SPF 11

| Part | Raw materials | INCI name | % (wt.) |
|---|---|---|---|
| A | Eumulgin VL 75 | Lauryl Glucoside | 3.00 |
| | | (and) Polyglyceryl- | |
| | | 2-Dipolyhydroxy- | |
| | | stearate (and) | |
| | | Glycerin | |
| | Tegosoft TN | C12-25 Alkyl Benzoate | 20.00 |
| | Copherol 1250 | Tocopherylacetat | 0.50 |
| | UV filter | | 3.00 |
| | compound according | | |
| | to formula I | | |
| | Perfume oil | Perfume (Fragrance) | 0.20 |
| | Neo Heliopan ® 303 | Octocrylene | 5.00 |
| | Carbopol 2984 | Carbomer | 0.35 |
| | Pemulen TR-1 | Acrylates/C10-30 | 0.15 |
| | | Alkylacrylate | |
| | | Crosspolymer | |
| B | Water, dist. | Water (Aqua) | 60.50 |
| | EDETA BD | Disodium EDTA | 0.10 |
| | Glycerol, 99% | Glycerin | 5.00 |
| | Phenoxyethanol | Phenoxyethanol | 0.70 |
| | Solbrol M | Methylparaben | 0.20 |
| | Solbrol P | Propylparaben | 0.10 |
| C | NaOH, 10% strength | Sodium Hydroxide | 1.20 |

Preparation process
Part A: Dissolve the UV absorber according to formula I in the oils or liquid UV filters (heat to approx. 70° C.). Allow to cool to approx. 30° C., add the remaining constituents apart from the Carbopol and Pemulen and mix at room temperature (stir for approx. 5 minutes). Stir in the Carbopol and Pemulen.
Part B: Dissolve the Solbrols in the phenoxyethanol, while heating. Mix with water and glycerol, add to part A, while stirring. Stir for approx. 60 minutes.
Part C: Add to A/B, homogenize with the Ultra Turrax.

### Recipe Example 6

Sunscreen Cream (W/O), In Vitro SPF 4, Water-Resistant

| Part | Raw materials | INCI name | % (wt.) |
|---|---|---|---|
| A | Dehymuls PGPH | Polyglyceryl-2 | 5.00 |
| | | Dipolyhydroxy- | |
| | | stearate | |
| | Copherol 1250 | Tocopherylacetat | 0.50 |
| | Permulgin 3220 | Ozokerite | 0.50 |
| | Zinc stearate | Zinc Stearate | 0.50 |
| | Tegosoft TN | C12-15 Alkyl | 25.00 |
| | | Benzoate | |
| | UV filter | | 5.00 |
| | compound | | |
| | according to | | |
| | formula I | | |
| B | Water, dist. | Water (Aqua) | 57.90 |
| | EDETA BD | Disodium EDTA | 0.10 |
| | Glycerol, 99% | Glycerin | 4.00 |
| | Phenoxyethanol | Phenoxyethanol | 0.70 |
| | Solbrol M | Methylparaben | 0.20 |
| | Solbrol P | Propylparaben | 0.10 |
| | Magnesium sulfate | Magnesium Sulfate | 0.50 |

Preparation process
Part A: Heat to approx. 85° C.
Part B: Heat to approx. 85° C. (without zinc oxide; disperse zinc oxide in with the Ultra Turrax). Add B to A. Allow to cool, while stirring, subsequently homogenize.

Sunscreen Soft Cream (W/O), In Vitro SPF 40

| Part | Raw materials | INCI name | % (wt.) |
|---|---|---|---|
| A | Dehymuls PGPH | Polyglyceryl-2 | 5.00 |
| | | Dipolyhydroxystearate | |
| | Copherol 1250 | Tocopherylacetat | 0.50 |
| | Permulgin 3220 | Ozokerite | 0.50 |
| | Zinc stearate | Zinc Stearate | 0.50 |
| | Tegosoft TN | C12-15 Alkyl Benzoate | 10.00 |
| | UV filter | | 2.00 |
| | compound according | | |
| | to formula I | | |
| | Neo Heliopan ® 303 | Octocrylene | 5.00 |
| | Neo Heliopan ® MBC | 4-Methylbenzylidene | 3.00 |
| | | Camphor | |
| | Zinc oxide neutral | Zinc Oxide | 5.00 |
| B | Water, dist. | Water (Aqua) | 62.90 |
| | EDETA BD | Disodium EDTA | 0.10 |
| | Glycerol, 99% | Glycerin | 4.00 |
| | Phenoxyethanol | Phenoxyethanol | 0.70 |
| | Solbrol M | Methylparaben | 0.20 |
| | Solbrol P | Propylparaben | 0.10 |
| | Magnesium sulfate | Magnesium Sulfate | 0.50 |
| C | Perfume oil | Perfume (Fragrance) | 0.20 |

Preparation process
Part A: Heat to approx. 85° C.
Part B: Heat to approx. 85° C. (without zinc oxide; disperse zinc oxide in with the Ultra Turrax). Add B to A. Allow to cool, while stirring.
Part C: Add and subsequently homogenize.

### Recipe Example 8

Sunscreen Milk (W/O)

| Part | Raw materials | INCI name | % (wt.) |
|---|---|---|---|
| A | Dehymuls PGPH | Polyglyceryl-2 | 3.00 |
| | | Dipolyhydroxystearate | |
| | Beeswax 8100 | Beeswax | 1.00 |
| | Monomuls 90-0-18 | Glyceryl Oleate | 1.00 |
| | Zinc stearate | Zinc stearate | 1.00 |
| | Cetiol SN | Cetearyl Isononanoate | 5.00 |
| | Cetiol OE | Dicaprylyl Ether | 5.00 |
| | Tegosoft TN | C12-15 Alkyl Benzoate | 4.00 |
| | Copherol 1250 | Tocopheryacetat | 0.50 |
| | Solbrol P | Propylparaben | 0.10 |
| | Neo Heliopan ® OS | Ethylhexyl Salicylate | 5.00 |
| | Neo Heliopan ® AV | Ethylhexyl Methoxy- | 7.50 |
| | | cinnamate | |
| | UV filter | | 1.50 |
| | compound | | |
| | according to | | |
| | formula I | | |
| B | Water, dist. | Water (Aqua) | 44.10 |
| | Trilon BD | Disodium EDTA | 0.10 |
| | Glycerol, 99% | Glycerin | 5.00 |
| | Solbrol M | Methylparaben | 0.20 |
| | Phenoxyethanol | Phenoxyethanol | 0.70 |
| | Neo Heliopan ® AP | Disodium Phenyl | 15.00 |
| | 10% strength solution | Dibenzimidazole | |
| | neutralized with NaOH | Tetrasulfonate | |
| C | Perfume oil | Perfume (Fragrance) | 0.30 |
| | Bisabolol | Bisabolol | 0.10 |

Preparation process
Part A: Heat to approx. 85° C.
Part B: Heat to approx. 85° C. Add B to A. Allow to cool, while stirring.
Part C: Add and subsequently homogenize.

### Recipe Example 9

Day Care Cream With UV Protection

| Part | Raw materials | INCI name | % (wt.) |
|---|---|---|---|
| A | Emulgade PL 68/50 | Cetearyl Glycoside (and) | 4.50 |
| | | Cetearyl Alcohol | |
| | Cetiol PGL | Hexyldecanol (and) | 8.00 |
| | | Hexyldecyl | |
| | | Laurate | |
| | Myritol 331 | Cocoglycerides | 8.00 |
| | Copherol 1250 | Tocopheryl Acetat | 0.50 |
| | Neo Heliopan ® E1000 | Isoamyl-p- | 2.00 |
| | | Methoxycinnamate | |
| | UV filter | | 2.00 |
| | compound according | | |
| | to formula I | | |
| B | Water, dist | Water (Aqua) | 45.40 |
| | Glycerol | Glycerin | 3.00 |
| | Phenonip | Phenoxyethanol (and) | 0.50 |
| | | Methylparaben (and) | |
| | | Butyparaben (and) | |
| | | Ethyparaben | |
| | | (and) Propylparaben | |
| C | Water, dist | Water (Aqua) | 25.00 |
| | Carbopol ETD 2050 | Carbomer | 0.20 |
| | NaOH, 10% strength | Sodium Hydroxide | 0.60 |
| D | Perfume oil | Perfume (Fragrance) | 0.30 |

Preparation process
Part A: Heat to 80° C.
Part B: Heat to 80° C. Add to part A, while stirring.
Part C: Disperse the Carbopol in the water and neutralize with sodium hydroxide solution. Add to part A/B at approx. 55° C.
Part D: Add at RT and homogenize.

### Recipe Example 10

Sunscreen Spray

| Part | Raw materials | INCI name | % (wt.) |
|---|---|---|---|
| A | Water, dem. | Water (Aqua) | 69.50 |
| | Glycerol, 99% | Glycerin | 4.00 |
| | 1,3-Butylene glycol | Butylene Glycol | 5.00 |
| | D-Panthenol | Panthenol | 0.50 |
| | Lara Care A-200 | Galactoarabinan | 0.25 |
| B | Baysilone oil M 10 | Dimethicone | 1.00 |
| | Edeta BD | Disodium EDTA | 0.10 |
| | Copherol 1250 | Tocopheryl Acetate | 0.50 |
| | Cetiol OE | Dicaprylyl Ether | 3.00 |
| | Neo Heliopan ® HMS | Homosalate | 5.00 |
| | Neo Heliopan ® AV | Ethylhexyl | 6.00 |
| | | Methoxycinnamate | |
| | Neo Heliopan ® 357 | Butyl Methoxydiben- | 1.00 |
| | | zoylmethane | |
| | UV filter compound | | 2.00 |
| | according to formula I | | |
| | alpha Bisabolol nat. | Bisabolol | 0.10 |
| | Pemulen TR-2 | Acrylates/C10-30 | 0.25 |
| | | Alkyl Acrylate | |
| | | Crosspolymer | |
| C | Phenoxyethanol | Phenoxyethanol | 0.70 |
| | Solbrol M | Methylparaben | 0.20 |
| | Solbrol P | Propylparaben | 0.10 |
| D | NaOH, 10% strength | Sodium Hydroxide | 0.60 |
| E | Perfume oil | Fragrance (Perfume) | 0.20 |

Preparation process
Part A: Dissolve the Lara Care A-200 in the other constituents of part A, while stirring.
Part B: Weigh out all the raw materials (without the Pemulen) and dissolve the crystalline substances, while heating. Disperse the Permulen in. Add part B to part A and homogenize for 1 minute.
Part C + D add and homogenize again with the Ultra Turrax for 1-2 minutes.

### Recipe Example 11

Sunscreen Hydrodispersion Gel (Balm)

| Part | Raw materials | INCI name | % (wt.) |
|---|---|---|---|
| A | Water, dist. | Water (Aqua) | 74.90 |
| | Carbopol 1342 | Acrylates/C10-30 | 1.00 |
| | | Alkyl Acrylate | |
| | | Crosspolomer | |
| | Triethanolamine | Triethanolamine | 1.20 |
| B | Neo Heliopan ® | Phenylbenzimidazole | 10.00 |
| | Hydro, 30% | Sulfonic Acid | |
| | strength solu- | | |
| | tion neutralized | | |
| | with TEA | | |
| C | Neo Heliopan ® AV | Ethylhexyl | 3.00 |
| | | Methoxycinnamate | |
| | UV filter | | 2.00 |
| | compound according | | |
| | to formula I | | |
| | Isopropyl myristate | Isopropyl Myristate | 4.00 |
| | Baysilone OIL PK 20 | Phenyl Trimethicone | 3.00 |
| | Phenonip | Phenoxyethanol (and) | 0.50 |
| | | Methylparaben (and) | |
| | | Butyparaben (and) | |
| | | Ethyparaben (and) | |
| | | Propylparaben | |
| | Perfume oil | Perfume (Fragrance) | 0.30 |
| | Bisabolol nat | Bisabolol | 0.10 |

Preparation process
Part A: Disperse the Carbopol in the water and neutralize with sodium hydroxide solution.
Part B: Add to part A, while stirring.
Part C: Dissolve the crystalline constituents in the other raw materials of part C, while heating (max. 40° C.), and add to part A/B. Stir thoroughly and subsequently homogenize. (Homozenta).

### Recipe Example 12

Hair Conditioner With UV Filters

| Part | Raw materials | INCI name | % (wt.) |
|---|---|---|---|
| A | Emulgade 1000 NI | Cetearyl Alcohol | 2.00 |
| | | (and) Ceteareth-20 | |
| | Lanette 16 | Cetyl Alcohol | 1.00 |
| | Neo Heliopan ® AV | 2 -Ethylhexyl | 3.00 |
| | | Methoxycinnamate | |
| | UV filter | | 1.00 |
| | compound according | | |
| | to formula I | | |
| B | Water, dist | Water (Aqua) | 91.70 |
| | Edeta BD | Disodium EDTA | 0.10 |
| | Phenonip | Phenoxyethanol (and) | 0.40 |
| | | Methylparaben (and) | |
| | | Butyparaben (and) | |
| | | Ethyparaben (and) | |
| | | Propylparaben | |
| | Dehyquart A-CA | Cetrimonium Chloride | 0.20 |
| | NaOH, 1% strength | Sodium Hydroxide | 0.30 |
| C | Perfume oil | Perfume (Fragrance) | 0.30 |

Preparation process
Part A: Heat to 80° C.
Part B: Heat to 80° C. Add to part A, while stirring.
Part C: Add at 40° C. and cool to RT.

### Recipe Example 13

Sunscreen Lotion (O/W)

| Part | Raw materials | INCI name | % (wt.) |
|---|---|---|---|
| A | Tegin M | Glyceryl Stearate | 2.50 |
| | Tagat S | PEG-30 Glyceryl Stearate | 1.95 |
| | Lanette O | Cetearyl Alcohol | 2.20 |
| | Hallbrite TQ | Diethylhexylnaphthalate | 7.00 |
| | Cetiol B | Dibutyl Adipate | 5.00 |
| | Tegosoft TN | C12-C15 Alkyl Benzoate | 4.00 |
| | Myritol PC | Propylene Glycol | 4.00 |
| | | Dicaprylate/Dicaprate | |
| | Phenonip | Phenoxyethanol (and) | 0.15 |
| | | Methylparaben (and) | |
| | | Butylparaben (and) | |
| | | Ethyl-paraben (and) | |
| | | Propylparaben | |
| | UV filter | | 2.00 |
| | compound according | | |
| | to formula I | | |
| | Neo Heliopan ® AV | Ethylhexyl | 5.00 |
| | | Methoxycinnamate | |
| B | Water, dist. | Water (Aqua) | 42.80 |
| | 1,2-Propylene glycol | Propylene Glycol | 2.00 |
| | Phenonip | Phenoxyethanol (and) | 0.30 |
| | | Methylparaben (and) | |
| | | Butylparaben (and) | |
| | | Ethylparaben (and) | |
| | | Propylparaben | |
| C | Water, dist. | Water (Aqua) | 19.00 |
| | Carbopol 2050 | Carbomer | 0.40 |
| | NaOH, 10% strength | Sodium Hydroxide | 1.70 |

Preparation process
Part A: Heat to 80-85° C.
Part B: Heat to 80-85° C., add part B to part A, while stirring.
Part C: Disperse the Carbopol in the water and neutralize with NaOH while stirring. Add part C at approx. 60° C., while stirring.

### Examples of Photostability Testing

Photostability tests were carried out with a Suntester from Heräus. The irradiation intensity in these was 80 W/m², based on the UV range of 290-400 nm. The irradiation time was 4 h in total, the photodegradation of the UV filters being measured by HPLC analyses after an irradiation time of 2 and 4 h. The irradiations of the UV filter mixtures were carried out in an isopropyl myristate solution. The percentage values relate to the value measured without irradiation (decrease in the concentration).

Comparison Measurement (Reference)

3% octyl methoxycinnamate (OMC)

1% 4-dimethylethyl-4′-methoxydibenzoylmethane (DMDBM)

| Time | OMC | DMDBM |
|---|---|---|
| 2 h | 58% | 67% |
| 4 h | 76% | 87% |

Compounds A1, A2 to be used according to the invention and compounds B1, B2 and B3 which are to be used not according to the invention are compared in the following.

Experiment 1 (According to the Invention):

3% octyl methoxycinnamate (OMC)

1% 4-dimethylethyl-4′-methoxydibenzoylmethane (DMDBM)

2% compound A1 (n-butyl ester), preferred compound of the formula I where
R¹ is methoxy which is located in the para position to the radical carrying the substituents R₄, R₅ and R₆,
R² and R³ are hydrogen,
R⁴ is CO₂R, where R is n-butyl
R⁵ is H and

R⁶ is phenyl

| | |
|---|---|
| | A1 |
| 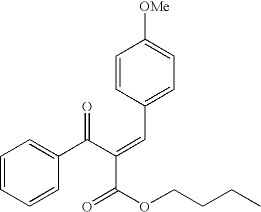 | |

| Time | A1 | OMC | DMDBM |
|---|---|---|---|
| 2 h | 0% | 12% | 24% |
| 4 h | 0% | 17% | 38% |

The investigation showed an outstanding photostability not only of the compound A1 itself, but also of the co-UV filters OMC and DMDBM, in comparison with the comparison measurement.

Note: Further experiments which are not described here in detail gave, for all the compounds (A₁-alkyl ester) where
R¹ is methoxy which is located in the para position to the radical carrying the substituents R₄, R₅ and R₆,
R² and R³ are hydrogen,
R⁴ is CO₂R, where R is C₁-C₅-alkyl,
R⁵ is H and
R⁶ is phenyl

similarly good photostability values.

Experiment 2 (According to the Invention)

3% octyl methoxycinnamate (OMC)

1% 4-dimethylethyl-4′-methoxydibenzoylmethane (DMDBM)

2% compound A2 (note: A2 is a solid)

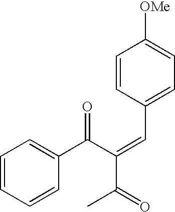

| Time | A2 | OMC | DMDBM |
|---|---|---|---|
| 2 h | 3% | 12% | 20% |
| 4 h | 3% | 17% | 28% |

Experiment 3 (According to the Invention)

3% octyl methoxycinnamate (OMC)

1% 4-dimethylethyl-4′-methoxydibenzoylmethane (DMDBM)

2% compound A3

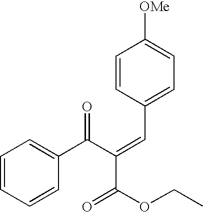

| Time | A2 | OMC | DMDBM |
|---|---|---|---|
| 2 h | 0% | 11% | 20% |
| 4 h | 0% | 17% | 28% |

Experiment 4 (Not According to the Invention)

3% octyl methoxycinnamate (OMC)

1% 4-dimethylethyl-4′-methoxydibenzoylmethane (DMDBM)

2% compound B1

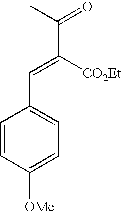

| Time | B1 | OMC | DMDBM |
|---|---|---|---|
| 2 h | 7% | 11% | 27% |
| 4 h | 12% | 18% | 48% |

Experiment 5 (Not According to the Invention):

3% octyl methoxycinnamate (OMC)

1% 4-dimethylethyl-4′-methoxydibenzoylmethane (DMDBM)

2% compound B2

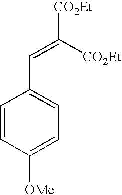

| Time | B2 | OMC | DMDBM |
|---|---|---|---|
| 2 h | 2% | 15% | 27% |
| 4 h | 7% | 30% | 60% |

Experiment 6 (Not According to the Invention):

3% octyl methoxycinnamate (OMC)

1% 4-dimethylethyl-4′-methoxydibenzoylmethane (DMDBM)

2% compound B3

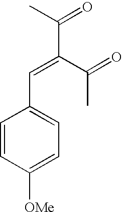

| Time | B3 | OMC | DMDBM |
|---|---|---|---|
| 2 h | 0% | 13% | 27% |
| 4 h | 8% | 25% | 52% |

### Examples of Solubility (In Per Cent By Weight)

| | Isopropyl | | |
|---|---|---|---|
| Substance | myristate | Miglyol -812 | Witconol-TN |
| A1 - Methyl ester | <10% | <10% | <10% |
| A1 - Ethyl ester | <10% | <10% | <10% |
| A1 - Isopropyl ester | >10% | >10% | >10% |
| A1 - n-Propyl ester | >10% | >10% | >10% |
| A1 - Isobutyl ester | >20% | >20% | >20% |
| A1 - n-Butyl ester | >20% | >20% | >20% |
| A1 - Isoamyl ester | >20% | >20% | >20% |
| A2 | <10% | <10% | <10% |
| A3 | >20% | >20% | >20% |

(data in per cent by weight)

For the structures of the compounds investigated, compare Experiments 1 - 3.

1. A method for enhancing the UV filtering effectiveness of a cosmetic formulation by adding to said formulation a compound of the formula 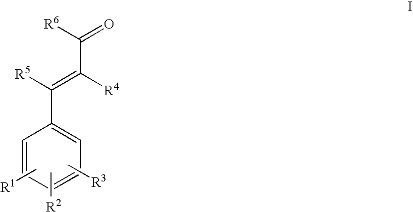
wherein
R¹-R³ independently of one another are hydrogen, C₁-C₈-alkyl or C₁-C₈-alkoxy,
R⁴ is COR, CO₂R, CONR₂, where R is C₁-C₈-alkyl or C₃-C₈-cycloalkyl
R⁵ is H or C₁-C₈-alkyl,
R⁶ is aryl, aryl substituted by up to three C₁-C₈-alkyl- or C₁-C₈-alkoxy, or C₃-C₈-cycloalkyl
as UV filters in cosmetic formulations, in particular in combination with UV filters from the group consisting of methoxycinnamate derivatives and/or dibenzoylmethane derivatives, with the proviso that R⁵ is H if R⁴ is COR.

2. A method according to claim 1, wherein
R¹-R³ independently of one another are hydrogen, C₁-C₈-alkyl or C₁-C₈-alkoxy,
R⁴is CO₂R, where R is C₁-C₈-alkyl,
R⁵is H or C₁-C₈-alkyl,
R⁶ is aryl or aryl substituted by up to three C₁-C₈-alkyl- or C₁-C₈-alkoxy.

3. A method according to claim 1, wherein
R¹ is methoxy,
R² and R³ are H,
R⁴ iS CO₂R, where R is C₂-C₅-alkyl and
R⁶ is phenyl or phenyl substituted by up to three C₁-C₈-alkyl- or C₁-C₈-alkoxy.

4. A method according to claim 1, wherein
R¹ is methoxy which is located in the para position to the radical carrying the substituents R₄, R₅ and R₆,
R² and R³ are H,
R⁴ is CO₂R, where R is C₄-C₅-alkyl,
R⁵ is H or C₁-C₈-alkyl, and
R⁶ is phenyl.

5. A method according to claim 1, wherein
R⁶ is C₃-C₈-cycloalkyl.

6. A method according to claim 5, wherein
R⁶ is cyclohexyl.

7. A method according to claim 5, wherein
R⁴ is CO₂R, where R is C₁-C₈-alkyl.

8. A method according to claim 5, wherein
R⁴is CO₂R, where R is ethyl.

9. A cosmetic or dermatological formulation, comprising
one or more compounds of the formula I as defined in claim 1, as UV absorbers.

10. Cosmetic or dermatological formulation according to claim 9, further comprising
(a) one or more further UV absorbers selected from the group consisting of methoxycinnamate derivatives and dibenzoylmethane derivatives, and/or
(b) coated or non-coated pigments of metal oxides, and
(c) mixtures thereof.

11. A cosmetic or dermatological formulation according to claim 10, wherein said UV absorbers and pigments employed are chosen and coordinated in their particular amount relative to one another such that they co-operate such that the sun protection factor of the formulation is increased synergistically.

12. A cosmetic or dermatological formulation according to claim 10, wherein said UV absorbers employed are chosen and coordinated in their particular amount relative to one another such that the critical wavelength of the formulation λ_{crit.} is >380 nm.

13. A compound of the formula (I), 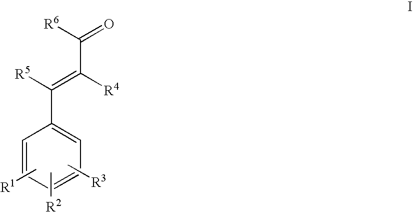
wherein
R¹ is C₁-C₈-alkoxy which is located in the para position to the radical carrying the substituents R₄, R⁵and R⁶,
R² and R³ are hydrogen,
R⁴ is CO₂R, where R is C₁-C₈-alkyl,
R⁵ is H,
R⁶ is phenyl or cyclohexyl.

14. A compound according to claim 13, wherein
R⁴ is CO₂R, where R is methyl, ethyl, n-propyl, iso-propyl, n-butyl, sec-butyl, iso-butyl, tert-butyl or iso-amyl and
R⁶ is phenyl.

15. A compound according to claim 14, wherein
R⁴is CO₂R, where R is methyl, ethyl, n-propyl, iso-propyl, n-butyl, sec-butyl, iso-butyl or tert-butyl and
R⁶ is cyclohexyl.
